(19) United States
(12) Patent Application Publication
(10) Pub. No.: US 2025/0088034 A1
(43) Pub. Date: Mar. 13, 2025

(54) METHOD AND SYSTEM FOR CHARGING AN ELECTRONIC MODULE

(71) Applicant: Sanofi, Paris (FR)

(72) Inventor: Florian Eberli, Wil (CH)

(21) Appl. No.: 18/727,614

(22) PCT Filed: Jan. 9, 2023
(86) PCT No.: PCT/EP2023/050259

§ 371 Date: Jul. 9, 2024

(30) Foreign Application Priority Data
Jan. 10, 2022 (EP) 22315008.7

(51) Int. Cl. H02J 50/12; A61M 5/31

(52) U.S. Cl. CPC H02J 50/12; A61M 5/31; A61M 2205/8243

(57) ABSTRACT

A method for wireless charging of an electronic module of a drug delivery device and to a respective charging system. The method includes the steps of connecting a power source with an oscillator of a charger station, wherein the charger station includes a transmitter side resonator circuit inductively coupled to the oscillator, placing an electronic module having a receiver side resonator circuit inductively coupled to a rectifier connected to a rechargeable battery in a distance from the charger station being equal or less than 15 cm, and tuning or matching the frequency of the transmitter side resonator circuit to the frequency of the receiver side resonator circuit to establish a resonant coupling between the transmitter side resonator circuit and the receiver side resonator circuit for transferring power from the charger station to the electronic module.

### CROSS-REFERENCE TO RELATED APPLICATIONS

This is a National Stage Application, filed under 35 U.S.C. § 371, of International Patent Application No. PCT/EP2023/050259, filed on Jan. 9, 2023, which claims priority to European Patent Application No. 22315008.7, filed on Jan. 10, 2022, the disclosures of which are incorporated herein by reference in their entireties.

### TECHNICAL FIELD

The present invention is generally directed to a method for wireless charging of an electronic module of a drug delivery device. The present invention further relates to a charging system for wireless charging of an electronic module of a drug delivery device.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament. However, setting the correct dose amount may be difficult or burdensome for, e.g., visually and/or manually impaired patients.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present invention is applicable for disposable and reusable devices.

For such devices the functionality of recording doses that are dialled and delivered from the pen may be of value to a wide variety of device users as a memory aid or to support detailed logging of dose history. Thus, drug delivery devices using electronics are becoming increasingly popular in the pharmaceutical industry as well as for users or patients. For example, a dose recording system is known from WO 2021/116387 A1 comprising a drug delivery device and an electronic module which is removably mechanically coupled to the drug delivery device. The electronic module of this known device is provided with a rechargeable battery (accumulator).

Wireless charging of electronic modules is generally known. Typically short range inductive coupling technologies, e.g., the QI-standard for wireless charging of smart phones, is applied. Devices that operate with the Qi standard rely on electromagnetic induction between planar coils. A Qi system consists of two types of devices, namely a base station, which is connected to a power source and provides inductive power, and a mobile device, which consumes inductive power. The base station contains a power transmitter that comprises a transmitting coil that generates an oscillating magnetic field. The mobile device contains a power receiver holding a receiving coil. The magnetic field induces an alternating current in the receiving coil (Faraday's law of induction). This power transfer is efficient if a close spacing of the two coils and a shielding on their surfaces is ensured. More specifically, the shielding is needed on the two “outer” faces of the coils, i.e., the faces which are directed away from the other coil.

In addition, power transfer by means of resonant circuits is known, e.g., from EP 3 544 196 A1. This technology is relatively new and requires coils with extremely low losses, i.e., with very high-Q resonance circuits, which result in high voltages and currents inside the resonant circuit. In addition, efficient power transfer requires tuning the resonance circuits to the exact same frequency, and to achieve this, manufacturing tolerances must be compensated. Due to limited space in electronic modules used in or with drug delivery devices, the resonant circuit on the receiver side can hardly be automatically tuned or matched to a known frequency. In addition to the further restrictions, the space required for a tuning circuit in an electronic module used in or with drug delivery devices precludes the use of the technology of loosely coupled resonant circuits in the field of pen-type drug delivery devices if the receiver circuit must be tuned.

### SUMMARY

It is an object of the present disclosure to further improve user-friendliness of wireless charging of an electronic module used in or with pen-type drug delivery devices.

This object is solved for example by the subject matter defined in the independent claims. Advantageous embodiments and refinements are subject to the dependent claims. However, it should be noted that the disclosure is not restricted to the subject matter defined in the appended claims. Rather, the disclosure may comprise improvements in addition or as an alternative to the ones defined in the independent claims as will become apparent from the following description.

One aspect of the disclosure relates to a method for wireless charging of an electronic module of a drug delivery device. This method comprises the provision of a charger station with an oscillator, e.g., a source coil through which alternating current (AC) electricity runs to form an oscillating electromagnetic field, connectable with a power source and with a transmitter side resonator circuit inductively coupled to the oscillator, and an electronic module with a rectifier, e.g., with a load coil, connected to a rechargeable battery (accumulator) and with a receiver side resonator circuit inductively coupled to the rectifier. Further, the method comprises the steps of connecting the power source with the oscillator of the charger station and placing the electronic module in a distance from the charger station being equal or less than 15 centimeters (cm), and matching, tuning or synchronizing the frequency of the transmitter side resonator circuit to the frequency of the receiver side resonator circuit. This will establish a resonant coupling between the transmitter side resonator circuit and the receiver side resonator circuit for transferring power from the charger station to the electronic module (tunneling).

The inductive coupling between the oscillator and/or the rectifier with the respective resonator circuit may be achieved by a respective coil, e.g., a single copper loop. The resonator circuit(s) may be a coil optionally connected to a capacitor. Preferably, the resistance in the resonator coils is significantly reduced, e.g., using a high frequency stranded wire of a sufficient diameter. Further, it is preferred that the optional capacitor is able to carry high voltages, e.g., of several 1.000 V.

Applying the tunneling technology based on resonant coupling has the benefit over classical short range inductive coupling technologies (e.g., QI-standard for wireless charging of smart phones) in that a much longer transmission range is possible. In other words, it is not required to place the electronic module directly adjacent or abutting the charger station. Rather, a distance of e.g., up to centimeters (cm) is sufficient for charging the battery of the electronic module. This facilitates charging and improves user-friendliness. In addition, resonant coupling provides a very high efficiency even in long range applications and does not radiate electromagnetic fields. The oscillating magnetic field generated by the resonance circuit is sinusoidal and nearly distortion-free which is a great benefit in terms of electromagnetic compatibility. Still further, the coils must not be strictly orientated. Rather, due to the tunneling effect a wide range of misalignment can be compensated.

According to the present disclosure, the frequency of the transmitter side resonator circuit is matched or tuned to the frequency of the receiver side resonator circuit which avoids bulky circuitry on the receiver side for automatically tuning and/or matching the resonant circuit on the receiver side to a known frequency of the transmitter. In an optional embodiment, frequency and phase may be matched, i.e., synchronized such that two existing signals are brought to an overlap. This may be the case in an option, where the receiving circuit is oscillating already by itself and the transmitter then has to match its signal to enable a resonant coupling. There are several options for matching the frequency or synchronizing the signal of a transmitting device to a receiver with a fixed resonance frequency which may be used as alternatives or in combination.

For example, multiple transmitter coils and resonator circuits may be provided in different spatial orientations. This could allow to always select the best one, i.e., the one with the best transmission ratio.

According to a first option, the step of tuning or matching the frequency of the transmitter side resonator circuit to the frequency of the receiver side resonator circuit comprises the steps of generating a frequency sweep on the transmitter side and measuring the power delivered by the transmitter side resonator circuit, e.g., detecting a load. Depending on the Q, i.e., the losses in the coils, of the resonance circuits and the initial coupling factor of both circuits, a long retention time might be needed on a frequency to stimulate the receiver up to a level which can be measured by the transmitter. Nevertheless, this first option provides a simple and reliable way for tuning adaptable transmitters to receivers with a fixed resonance frequency.

Further, according to a second option, the step of tuning or matching the frequency of the transmitter side resonator circuit to the frequency of the receiver side resonator circuit comprises the steps of generating a frequency sweep on the transmitter side and wirelessly transmitting an answer from the electronic module to the charger station, e.g., via Bluetooth, if the receiver side resonator circuit has received an amount of energy. This approach speeds up the tuning or matching procedure.

Still further, according to a third option, the step of tuning or matching the frequency of the transmitter side resonator circuit to the frequency of the receiver side resonator circuit comprises the steps of frequently stimulating the coil of the receiver side resonator circuit by means of the electronic module, receiving this signal by the charger station, e.g., by a wideband antenna, measuring its phase and frequency and tuning the transmitter side resonator circuit onto said phase and frequency of the receiver side resonator circuit. In other words, the coil on the receiver could frequently be stimulated by the receiver itself which could generate an oscillating magnetic field corresponding to the resonance frequency of the receiver. This may include the mismatch in frequency caused by foreign objects. The transmitting unit could receive this signal by a wideband antenna which could be a resonance circuit with low Q and measure its phase and frequency. This information could be used to tune the resonant circuit on the transmitting device side onto the exact phase and frequency of the receiver. This approach offers an even faster tuning time and has the additional benefit of being very precise and efficient. Further, this method allows to establish the tunnel effect from the beginning offering the advantage of compensating coplanarity misalignment of the coils up to a relatively wide angle.

Generally, for these options it is not required that the receiver has to know its exact resonance frequency. Rather, the transmitter is responsible for identifying the relevant frequency at which a resonant coupling and power transfer can occur, and to adapt its resonance circuit accordingly. In more detail, according to an example of the present invention, the working principle may comprise the following steps:

### Step 1:

The receiver stimulates its load coil by a short signal impulse with broad frequency content.

Examples of such a stimulation signal may include a chirp signal, i.e., a sinusoidal wave with increasing frequency, starting at, e.g., 0 Hz or higher. For the purpose of the stimulation, the chirp signal does not have to start at 0 Hz. A frequency sweep limited to the expected range of potential resonance frequencies can be used instead.

### Step 2:

The stimulation signal is inductively coupled from the receiver's load coil to the receiver side resonance circuit.

### Step 3:

A “ringing” effect occurs in the receiver resonance circuit, i.e., frequencies away from the resonance frequency are quickly damped, and the circuit continues to briefly oscillate at its resonance frequency (i.e., depending on its Q-factor). The apparent resonance frequency includes effects of foreign objects which are present in the vicinity of the coil.

Optional step 3a:

The receiver estimates the resonance frequency of its resonator based on an observation of the impedance change at its load coil. It then repeats steps 1-3, but with a stimulation signal just near and around the observed resonance frequency, resulting in a stronger and more efficient stimulation of the receiver side resonator (assuming the same amount of energy is used).

### Step 4:

The wide-band antenna on the transmitter side is used to detect this (weak) “ringing” signal (frequency and phase).

### Step 5:

The transmitter side matches its resonance frequency to the detected signal and starts to stimulate its resonance circuit in phase with the received signal.

### Step 6:

Energy starts to build up in the resonance circuit of the transmitter and starts to be transferred to the receiver as well, gradually building up the resonant coupling.

A frequency sweep may be used to detect the frequency at which the power transfer is most efficient. Here, it is not important that this frequency must be strictly known. The practical goal of the method is rather to detect the frequency at which the power transfer is most efficient. This frequency will depend also on parasitic and detuning effects generated by nearby objects and materials, and on the load applied by the electronic module that is being charged. Also, the frequency can change over time. Thus, instead of the actual resonance frequency of the receiver any reference to the resonance frequency may be understood as reference to the system resonance frequency which is useful to achieve the intended resonant coupling effect and an efficient power transmission.

According to a further aspect of the present disclosure, the electronic module is placed in a distance from the charger station being equal or less than 12 centimeters (cm), e.g., in a distance from the charger station being equal or less than 3 to 6 cm. This allows a much simpler handling compared to the classical short range inductive coupling technologies (e.g., QI-standard for wireless charging of smart phones) as the electronic module or the drug delivery device comprising the electronic module has to be placed in the vicinity of the charger station without aligning these units to each other or without taking care of placing these units very closely next to each other. For example, the receiver side coil may have a diameter of 15 mm, and the electronic module may be placed in a distance from the charger station being equal or less than ten times, preferably equal or less than four to eight times, the coil diameter of the receiver side resonator circuit.

According to an independent aspect of the present disclosure, a charging system for an electronic module of a drug delivery device may comprise a charger station with an oscillator connectable with a power source and an electronic module with a rectifier connected to a rechargeable battery. The charging system may further comprise a pair of resonance circuits having a high Q factor with a transmitter side resonator circuit coupled inductively to the oscillator in the charger station and a receiver side resonator circuit coupled inductively to the rectifier in the electronic module. The quality factor or Q factor is a dimensionless parameter that describes the damping or rate of energy loss in an oscillator. A high Q factor corresponds to low losses. It is also approximately defined as the ratio of the energy stored in the resonator to the energy lost in one radian of the cycle of oscillation. The Q factor may alternatively be defined as the ratio of a resonator's center frequency to its bandwidth when subject to an oscillating driving force. Low losses are desirable to get the required strong resonant oscillation, and the reduction of the bandwidth is relevant to the tuning process. For transferring power from the charger station to the electronic module the charger station and the electronic module are configured to establish a resonant coupling between the transmitter side resonator circuit and the receiver side resonator circuit, e.g., according to the above described method.

The charging system may comprise at least one resonance circuit having a Q factor of at least 500, e.g., at least 800, preferably of at least 1.000. In an embodiment, the Q factor may be in a range between 800 and 1.200.

In addition or as an alternative, at least one resonator circuit may be formed by a single layer solenoid, i.e., a one loop coil, in parallel with a capacitor. For example, the transmitter side resonator circuit and/or the receiver side resonator circuit is formed by a single layer solenoid in parallel with a capacitor.

For transferring power from the charger station to the electronic module the transmitter side resonator circuit in the charger station and the receiver side resonator circuit in the electronic module may have substantially the same or at least a very similar resonant frequency.

In the charging system, the electronic module may be permanently or releasably attached or integrated in a drug delivery device for setting and dispensing variable doses of a liquid drug. For example, the drug delivery device comprises a cartridge containing a liquid drug and a dose setting and drive mechanism which is configured to perform a dose dialing operation for selecting a dose to be delivered by the drug delivery device and a dose delivery operation for delivering the set dose. The electronic module may comprise at least one sensor configured to detect operation of the dose setting and drive mechanism and a processor configured to control operation of the at least one sensor and to process and/or store signals from the at least one sensor. For example, the at least one sensor may be an optical sensor for detecting a dose delivery operation of the dose setting and drive mechanism.

The electronic module may further comprise a communication unit for wireless communicating with another device, e.g., for wireless communicating with the charger station and/or a mobile phone. The communication unit may comprise a wireless communication interface for communicating with another device via a wireless network such as Wi-Fi or Bluetooth, or even an interface for a wired communications link, such as a socket for receiving a Universal Serial Bus (USB), USB-C, mini-USB or micro-USB connector. Preferably, the electronic module comprises an RF, WiFi and/or Bluetooth unit as the communication unit. The communication unit may be provided as a communication interface between the electronic module and the exterior, such as other electronic devices, e.g., mobile phones, personal computers, laptops and so on. For example, dose data may be transmitted by the communication unit to the external device. The dose data may be used for a dose log or dose history established in the external device. In addition or as an alternative, the communication unit may receive data from the external device, e.g., data regarding a health condition of a user and/or dose data regarding an amount of drug to be delivered by the drug delivery device.

According to a still further aspect of the present disclosure, a drug delivery device is provided which may comprise or may be coupled to an electronic module, wherein the electronic module is configured such that its rechargeable battery may be charged by means of loosely coupled resonant circuits.

The drug delivery device may be a reusable device permitting replacement of an empty cartridge. For example, the cartridge may be received in a releasably attached cartridge holder.

In one embodiment, the drug delivery device comprises a dial sleeve, e.g., a number sleeve, which is rotatable relative to a housing, e.g., along a helical path, at least in the dose setting operation. In addition, a manually operable injection trigger, for example, a dose and/or injection button or a member axially and/or rotationally locked thereto may be axially displaceable relative to the dial sleeve and rotationally constrained to the housing at least in the dose delivery operation.

The present disclosure further pertains to a drug delivery device with the electronic module, e.g., a dose recording module as disclosed in WO 2021/116387 A1, for use with a charging system as described above which drug delivery device comprises a cartridge containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(o-carboxyheptadecanoyl)-des(B30) human insulin and B29-N-(o-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g., the axis which extends through the proximal and distal ends of the cartridge, the cartridge holder or the drug delivery device.

### BRIEF DESCRIPTION OF THE DRAWINGS

Non-limiting, exemplary embodiments of the disclosure will now be described with reference to the accompanying drawings, in which:

FIG. 1 shows a perspective view of a charging system according to the present invention; and

FIG. 2 is a schematic illustration of the resonant inductive coupling working principle of the charging system of FIG. 1.

In the figures, identical elements, identically acting elements or elements of the same kind may be provided with the same reference numerals.

### DETAILED DESCRIPTION

In the following, some embodiments will be described with reference to an insulin injection device. The present disclosure is however not limited to such application and may equally well be deployed with injection devices that are configured to eject other medicaments or drug delivery devices in general, preferably pen-type devices and/or injection devices.

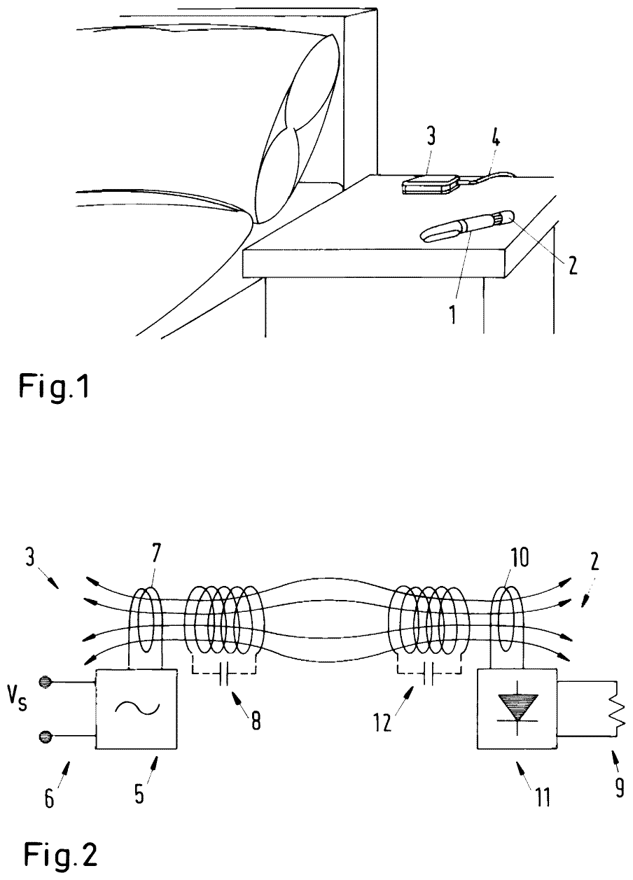

FIG. 1 shows a pen-type drug delivery device 1 with an electronic module 2 which may be attached to or integrated into the drug delivery device 1. In the depicted exemplary embodiment, the electronic module 2 is arranged at the proximal end, i.e., the end facing away from an injection needle, of the drug delivery device 1. The electronic module 2 may be fully integrated, e.g., in a button or knob of the drug delivery device 1, or may be permanently or releasably attached to the drug delivery device 1, e.g., as a cap connectable with a button or knob of the drug delivery device 1.

In addition, a charger station 3 is depicted spaced a few centimeters (cm) from the drug delivery device 1. The charger station 3 may be connected to a power source via cable 4.

The working principle of transferring energy from the charger station 3 to the electronic module 2 by means of resonant inductive coupling is shown in FIG. 2 in which the charger station 3 acting as an energy transmitter is schematically depicted on the left side and the electronic module 2 being the energy receiver is schematically depicted on the right side. The charger station 3 comprises an oscillator 5 connectable with a power source 6. A source coil 7 of the oscillator 5 is inductively coupled to a transmitter side resonator circuit 8.

In a similar manner, a load, e.g., a rechargeable battery 9, of the electronic module 2 is connected with a load coil 10 via a rectifier 11, wherein the load coil 10 is inductively coupled to a receiver side resonator circuit 12.

Source coil 7 and load coil 10 are depicted in the example of FIG. 2 as comprising several loops but may be a single copper loop, each.

The transmitter side resonator circuit 8 and the receiver side resonator circuit 12 each comprise a selfresonant coil. The coils of the transmitter side resonator circuit 8 and the receiver side resonator circuit 12 resonate at the same frequency. For example, the resonant circuits 8, 12 are coils of copper wire which resonate with their internal capacitance (shown as dotted capacitors in FIG. 2) at, e.g., 10 MHz.

When connected to the power source 6, an alternating current (AC) electricity runs through source coil 7 within charger station 3 to form an oscillating electromagnetic field such that the transmitter side resonator circuit 8 may output a sine wave with a frequency of, e.g., 10 MHz. This frequency is not limited to 10 MHz but is rather to be adapted to the frequency at which the transmitter side resonator circuit 8 and the receiver side resonator circuit 12 resonate. The receiver side resonator circuit 12 resonating at the same frequency as the transmitter side resonator circuit 8 captures the field's energy and a rectifier delivers direct current (DC) to battery 9.

Assuming an electronic module 2 in a pen 1 cap containing a coil 12 with a diameter of 15 mm, a transmission distance of more than 3 cm, e.g., 6 cm to 12 cm, might be achieved. With a bigger diameter on the transmitter side this distance might be further increased to, e.g., 15 cm. It is not required that both coils have the same diameter.

In use, assuming a very high Q factor, i.e., low losses, the energy stored in the resonant circuit builds up over multiple cycles which leads to an intense level in the magnetic field in between both resonators 8, 12. The increasing power in the resonating field leads to the capability of transferring power over a wider distance. The receiving coil 10 in the end only receives a percentage of that energy but profits from the high energy and the resulting tunneling effect of the resonating field.

Further, the transmitting oscillator and the receiver circuit may cause a mismatch of the resonance circuits 8, 12 which is dependent on the load. Thus, it may be desired that the impedance of the load is matched to increase the efficiency and to keep the resonance frequency the same on both sides.

The above mentioned tunneling effect is only present when both circuits are oscillating substantially in phase. The critical moment is the startup of the receiving coil 12 where the transferred energy must be sufficient to stimulate the resonant circuit and to bring up the resonating current in the resonator. After the startup, when both circuits are in resonance, the tunneling effect will lead to higher efficiency and lower losses.

The resistance in the coils should be reduced as far as possible, e.g., by using a high frequency stranded wire of a sufficient diameter for the coils 7, 8, 10 and/or 12.

For synchronizing, tuning or matching the receiver side resonator circuit 12 with a fixed resonance frequency to the, e.g., autotuned, adaptive, transmitter side resonator circuit 8, a frequency sweep on the transmitter side may be generated and the power delivered by the transmitter can be measured. In addition or as an alternative, a frequency sweep can be generated with the transmitting coil and the receiving device could answer by, e.g., Bluetooth if the receiver circuit has received a small amount of energy. This requires a Bluetooth interface on the charger side but offers the capability to communicate the charging state or the current needed by the receiver. This approach speeds up the tuning or matching. Still further, the coil on the receiver may frequently be stimulated by the receiver itself which could generate an oscillating magnetic field corresponding to the resonance frequency of the receiver including the mismatch in frequency caused by foreign objects. The transmitting unit receives this signal by a wideband antenna and measure its phase and frequency. This information may be used to tune the resonant circuit on the transmitting device side onto the exact phase and frequency of the receiver. This approach offers an even faster tuning or matching time and it is very precise and efficient. Further the tunnel effect may exist from the beginning offering the advantage of compensating coplanarity misalignment of the coils up to a relatively wide angle.

The active resonator on the receiver side may also be used for the communication of information from the device to the power transmitter. This could be done by frequency modulation of the resonance circuit.

### REFERENCE NUMERALS

1 drug delivery device
2 electronic module
3 charger station
4 cable
5 oscillator
6 power source
7 source coil
8 transmitter side resonator circuit
9 rechargeable battery (accumulator)
10 load coil
11 rectifier
12 receiver side resonator circuit

1. A method for wireless charging of an electronic module of a drug delivery device, the method comprising:
connecting a power source with an oscillator of a charger station, wherein the charger station comprises a transmitter side resonator circuit inductively coupled to the oscillator,
placing the electronic module comprising a receiver side resonator circuit inductively coupled to a rectifier connected to a rechargeable battery in a distance from the charger station being equal or less than 15 cm,
tuning a frequency of the transmitter side resonator circuit to a frequency of the receiver side resonator circuit to establish a resonant coupling between the transmitter side resonator circuit and the receiver side resonator circuit for transferring power from the charger station to the electronic module.

2. The method according to claim 1, wherein the step of tuning the frequency of the transmitter side resonator circuit to the frequency of the receiver side resonator circuit comprises the steps of generating a frequency sweep on a transmitter side and measuring the power delivered by the transmitter side resonator circuit.

3. The method according to claim 1, wherein the step of tuning the frequency of the transmitter side resonator circuit to the frequency of the receiver side resonator circuit comprises the steps of generating a frequency sweep on a transmitter side and wirelessly transmitting an answer from the electronic module to the charger station after the receiver side resonator circuit (12) has received an amount of energy.

4. The method according to claim 1, wherein the step of tuning the frequency of the transmitter side resonator circuit to the frequency of the receiver side resonator circuit comprises the steps of frequently stimulating a coil of the receiver side resonator circuit by means of the electronic module, receiving this signal by the charger station, measuring its phase and frequency and tuning the transmitter side resonator circuit onto said phase and frequency of the receiver side resonator circuit.

5. The method according to claim 1, wherein the electronic module is placed in a distance from the charger station being equal or less than 12 cm.

6. The method according to claim 1, wherein the electronic module is placed in a distance from the charger station being equal or less than 6 cm.

7. The method according to claim 1, wherein the electronic module is placed in a distance from the charger station being equal or less than ten times a coil diameter of the receiver side resonator circuit.

8. A charging system for an electronic module of a drug delivery device, the charging system comprising:
a charger station comprising an oscillator connectable with a power source; and
an electronic module comprising a rectifier connected to a rechargeable battery,
wherein the charging system further comprises a pair of high-Q resonance circuits with a transmitter side resonator circuit inductively coupled to the oscillator in the charger station and a receiver side resonator circuit inductively coupled to the rectifier in the electronic module,
and wherein the charger station and the electronic module are configured to establish a resonant coupling between the transmitter side resonator circuit and the receiver side resonator circuit for transferring power from the charger station to the electronic module.

9. The charging system according to claim 8, wherein the pair of high-Q resonance circuits have a Q factor of at least 800.

10. The charging system according to claim 8, wherein the transmitter side resonator circuit is formed by a single layer solenoid in parallel with a capacitor.

11. The charging system according to claim 8, wherein the receiver side resonator circuit is formed by a single layer solenoid in parallel with a capacitor.

12. The charging system according to claim 8, wherein the transmitter side resonator circuit in the charger station and the receiver side resonator circuit in the electronic module have a same resonant frequency.

13. The charging system according to claim 8, wherein the electronic module is permanently or releasably attached or integrated in a drug delivery device for setting and dispensing variable doses of a liquid drug, wherein the drug delivery device comprises a cartridge containing a liquid drug and a dose setting and drive mechanism which is configured to perform a dose dialing operation for selecting a dose to be delivered by the drug delivery device and a dose delivery operation for delivering the set dose, and wherein the electronic module comprises at least one sensor configured to detect operation of the dose setting and drive mechanism and a processor configured to control operation of the at least one sensor and to process and/or store signals from the at least one sensor.

14. The charging system according to claim 13, wherein the at least one sensor is an optical sensor for detecting the dose delivery operation of the dose setting and drive mechanism.

15. The charging system according to claim 8, wherein the electronic module further comprises a communication unit for wirelessly communicating with another device.

16. The method according to claim 3, wherein the answer is wirelessly transmitted from the electronic module to the charger station via Bluetooth over a coupling.

17. The method according to claim 4, wherein the signal received by the charger station is by a wideband antenna.

18. The method according to claim 7, wherein the electronic module is placed in a distance from the charger station being equal or less than four to eight times the coil diameter of the receiver side resonator circuit.

19. The charging system according to claim 8, wherein the pair of high-Q resonance circuits have a Q factor of at least 1.000.

20. The charging system according to claim 8, wherein the electronic module further comprises a communication unit for wirelessly communicating with the charger station.
